(19) United States
(12) Patent Application Publication
(10) Pub. No.: US 2017/0202484 A1
(43) Pub. Date: Jul. 20, 2017

(54) WEARABLE DEVICE AND METHOD

(71) Applicant: Umm Al-Qura University, Makkah (SA)

(72) Inventors: Ali Mohammed Husain AL-SHAERY, Jeddah (SA); Raed Abdulrahman Khalil Shalwala, Makkah (SA)

(73) Assignee: Umm Al-Qura University, Makkah (SA)

(21) Appl. No.: 15/086,741

(22) Filed: Mar. 31, 2016

Related U.S. Application Data
(60) Provisional application No. 62/279,972, filed on Jan. 18, 2016.

(51) Int. Cl. A61B 5/11; H04W 4/02; H04W 4/00; A61B 5/00; A61B 5/08; A61B 5/01; A61B 5/021; A61B 5/145; H04B 1/3827; A61B 5/024

(52) U.S. Cl. CPC A61B 5/1112; H04B 1/385; H04W 4/025; H04W 4/005; A61B 5/024; A61B 5/08; A61B 5/01; A61B 5/021; A61B 5/14532; A61B 5/7475; A61B 5/6801; A61B 5/0022; A61B 5/742

(57) ABSTRACT

A wearable device, including a fastening device for fastening the wearable device to a user and a housing coupled to the fastening device. The housing includes a graphical display including a tactile user interface, a locationing system, communication circuitry, a sensor and processing circuitry. The processing circuitry is configured to obtain a location of the wearable device, transmit the location to a server, and receive guidance information from the server based on the location. The processing circuitry is also configured to determine a recommendation corresponding to the guidance information and output the recommendation. The processing circuitry is further configured to obtain a measured value of a physiological parameter via the sensor, determine a comparison between the measured value and a predetermined threshold, generate a feedback based upon the comparison, and output the feedback.

### CROSS-REFERENCE TO RELATED APPLICATIONS

This application is based on, and claims the benefit of priority to, provisional application No. 62/279,972, filed Jan. 18, 2016, the entire contents of which are incorporated herein by reference.

### FIELD

The present invention relates generally to a wearable device, and more particularly to a device and method for a wearable device that provides information corresponding to locational and physiological data.

### BACKGROUND

The “background” description provided herein is for the purpose of generally presenting the context of the disclosure. Work of the presently named inventor, to the extent it is described in this background section, as well as aspects of the description which may not otherwise qualify as prior art at the time of filing, are neither expressly nor impliedly admitted as prior art against the present disclosure.

The use of GPS as a positioning tool has been widely investigated and implemented by present studies and inventions. However, the transmission of GPS signals can be hindered in areas of high rise buildings, heavy canopy, high mountains and tunnels. As such, the use of GPS for collecting travel data is insufficient in arbitrary locations that may contain structures that deny the successful application of GPS location determination protocols.

Currently, standard off-the-self GPS devices can be cumbersome to use and carry. The tendency for travelers to utilize GPS devices is reduced as the dimensions of the devices become a burden as opposed to a convenient tool to aid in navigation and direction. Therefore, it would be desirable for a device to be developed to overcome the shortcomings of GPS utilization and to provide travelers with comfortably, continuous, and precise feedback as they navigate all types of areas and locations.

### SUMMARY

In an exemplary aspect, a wearable device, including a fastening device for fastening the wearable device to a user and a housing coupled to the fastening device. The housing includes a graphical display including a tactile user interface, a locationing system, communication circuitry, a sensor and processing circuitry. The processing circuitry is configured to obtain a location of the wearable device, transmit the location to a server, and receive guidance information from the server based on the location. The processing circuitry is also configured to determine a recommendation corresponding to the guidance information and output the recommendation. The processing circuitry is further configured to obtain a measured value of a physiological parameter via the sensor, determine a comparison between the measured value and a predetermined threshold, generate a feedback based upon the comparison, and output the feedback.

The foregoing general description of exemplary implementations and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an exemplary illustration of a wearable device system, according to certain aspects;

FIG. 2 is an exemplary illustration of a wearable device workflow, according to certain aspects;

FIG. 3 is an exemplary illustration of a wearable device system workflow, according to certain aspects;

FIG. 4 illustrates a navigation feedback process, according to certain exemplary aspects;

FIG. 5 illustrates a health monitoring process, according to certain exemplary aspects;

FIG. 6 illustrates a hardware block diagram of a wearable device, according to certain exemplary aspects;

FIG. 7 illustrates a hardware block diagram of a server, according to certain exemplary aspects;

FIG. 8 illustrates a hardware block diagram of a data processing system, according to certain exemplary aspects; and

FIG. 9 illustrates a hardware block diagram of a CPU, according to certain exemplary aspects.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

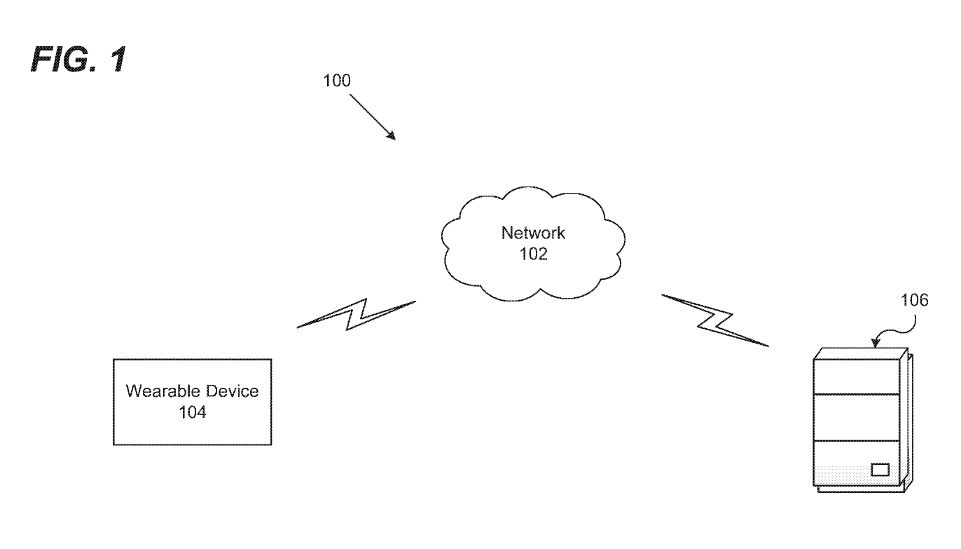

FIG. 1 is an exemplary illustration of a wearable device system 100, according to certain aspects. The wearable device system 100 includes a wearable device 104, a server 106 and a network 102. The wearable device system 100 enables communication between a wearable device 104 and a server 106 in which feedback is provided to the wearable device 104 based on data corresponding to detected locational information and measured physiological information of the wearable device 104.

The wearable device 104 represents one or more wearable devices 104, and is connected to the server 106 via the network 102. The wearable device 104 can include a watch, an armband, a wristband, and the like. The wearable device 104 can be configured to wirelessly connect to other wearable devices 104 within an area, to monitor the location of individuals and connect groups of people. Additionally, the wearable device 104 can employ a master-slave topology where the server 106 intelligently tracks and issues commands to a plurality of wearable devices 104. In certain aspects, the server 106 can be a wearable device 104 in which the wearable device 104 acts as a master wearable device to provide tracking and the issuance of commands to a slave wearable device.

The wearable device 104 can include a fastening device for fastening the wearable device 104 to a user and a housing coupled to the fastening device. The housing can include a graphical display, a locationing system, communication circuitry, a sensor, an output device and a processor. The graphical display can utilize a tactile user interface and be in communication with the processor of the wearable device 104. The locationing system can include one or more locationing systems such as Global Navigation Satellite Systems (GNSS), Global Positioning System (GPS), Wi-Fi, GALELIO, Geographic Information Systems (GIS), Global System for Mobile Communications (GSM), Inertial Navigation System (INS), and the like. The communication circuitry enables the wearable device 104 to communicate with the server 106 as well as other wearable devices 104. The sensor can include one or more sensors and can be utilized to measure a physiological parameter of the user. The output device can include one or more output devices such as an audio jack, a speaker, a haptic device, and the like, to provide additional feedback to the user of the wearable device 104. The processor includes circuitry which can be configured to determine feedback corresponding to detected locational information and measured physiological information.

The circuitry of the processor can be configured to obtain a location of the wearable device 104 via the locationing system, and transmit the location to the server 106. The circuitry can also be configured to receive guidance information from the server 106 based on the location of the wearable device 104. The circuitry can further be configured to determine a recommendation corresponding to the guidance information and output the recommendation via the graphical display. Exemplary aspects of the guidance information and the recommendation will be discussed further herein.

The circuitry of the processor can also be configured to obtain a measured value of a physiological parameter via the sensor and determine a comparison between the measured value and a predetermined threshold of the physiological parameter. The circuitry can further be configured to generate a feedback based on the comparison and out put the feedback via the graphical display. Exemplary aspects of the comparison and the feedback will be discussed further herein.

The server 106 represents one or more servers 106, and is connected to the wearable device 104 via the network 102. The server 106 includes circuitry configured to receive a location of the wearable device 104 via the communication circuitry and generate guidance information based on the location. The circuitry of the server 106 can also be configured to transmit other information and instructions to the wearable device 104. For example, the information and instructions may correspond to accidents and/or warnings that denote incidents which have occurred at a particular location. The particular location can be within proximity of the guidance information and result in the generation and transmission of alternate routes of navigation from the server 106 to the wearable device 104.

The network 102 represents one or more networks 102, and is connected to the wearable device 104 and the server 106. The network 102 can communicate via wired networks such as Ethernet, LAN or any other wired form of communication that is known. The network 102 can also communicate via wireless networks such as Wi-Fi, BLUETOOTH, cellular networks including EDGE, 3G and 4G wireless cellular systems, Infrared or any other wireless form of communication that is known.

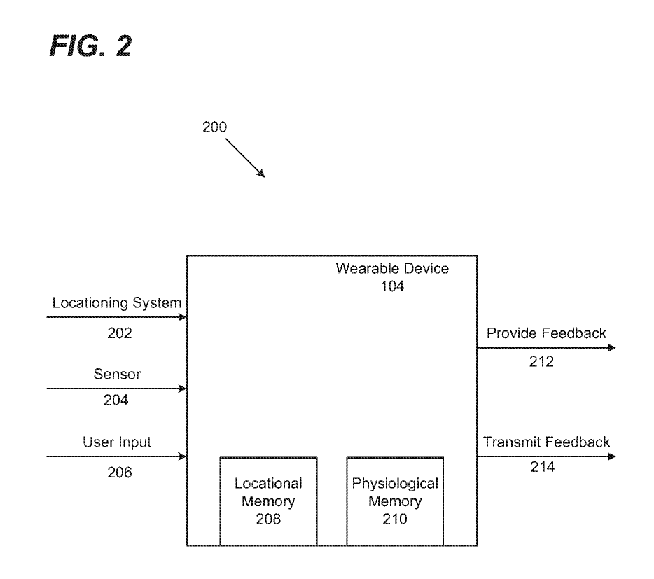

FIG. 2 is an exemplary illustration of a wearable device workflow 200, according to certain aspects. The wearable device workflow 200 describes a wearable device 104 that takes location and physiological information as an input and generates feedback as an output corresponding to the location and physiological information. The wearable device workflow 200 further describes how the wearable device 104 interacts with an environment the wearable device 104 is located in, such as interacting with other wearable devices 104.

The wearable device 104 includes a processor including circuitry that can receive location information via a locationing system 202. The locationing system 202 can be located in the housing of the wearable device 104. The locationing system 202 can determine an indoor location and an outdoor location of the wearable device 104 via at least one of GNSS, GPS, GIS, GALELIO, Wi-Fi, INS, GSM, and the like. The circuitry of the wearable device 104 can receive physiological information via a sensor 204 located in the housing of the wearable device 104. The sensor 204 can include one or more sensors 204 such as a heart rate sensor, a respiration rate sensor, a temperature sensor, a blood sugar sensor, a blood pressure sensor, and the like. The circuitry can further receive information via a user input 206. The user input 206 can be provided at the graphical display of the wearable device 104 via a tactile user interface of the graphical display. The user input 206 can include directional information, points of interest, a request of directions, physiological parameter inputs, and the like.

The circuitry of the processor can also be configured to store the inputs and/or feedback in memory. In some aspects, the memory is separated for locational information and for physiological information. For example, the circuitry can be configured to store the location of the wearable device 104 in locational memory 208 and the measured values of physiological parameters in physiological memory 210. In other aspects of the present disclosure, all information received as an input by the circuitry of the wearable device 104 can be stored in a single memory location.

The circuitry of the wearable device 104 can also be configured to provide feedback 212 based on the locational information and the physiological information. The provided feedback 212 based on the locational information can include a navigation to a predetermined location and navigation to a desired location that is received as an input at via the tactile interface of the graphical display. In other aspects, the provided feedback 212 based on the locational information can include a navigation recommendation based on a warning corresponding to an incident that has occurred at the predetermined location and/or the desired location. On the other hand, the provided feedback 212 can correspond to the physiological information in that the feedback includes a comparison of the measured physiological parameter to a predetermined threshold. For example, the feedback can include a warning when the measured physiological parameter does not satisfy the conditions of the predetermined threshold corresponding to the physiological parameter.

In some aspects of the present disclosure, the provided feedback 212 can be output to the user of the wearable device 104 via the graphical display. On other aspects, the feedback can be provided via an output device that is coupled to the housing of the wearable device 104. The output device can include one or more output devices such as an audio jack, a speaker, a haptic device and the like.

The circuitry of the wearable device 104 can further be configured to transmit feedback 214 based on the locational information and the physiological information to a remote device. The remote device can include a server 106, another wearable device, and the like. The transmission of feedback 214 to a remote device can allow for location information and physiological information to update in real time. Additionally, the transmission of the feedback 214 can enable the location the wearable device 104 to be precisely monitored and continuously aided in navigation.

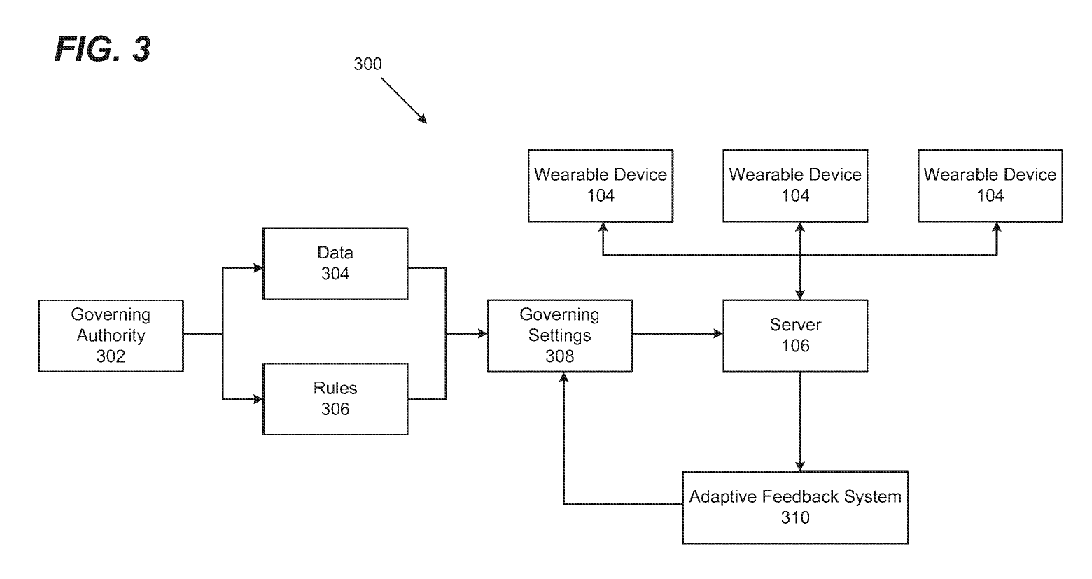

FIG. 3 is an exemplary illustration of a wearable device system workflow 300, according to certain aspects. The wearable device system workflow 300 describes an implementation of employing a master-slave topology where a server 106 intelligently tracks and issues commands to a plurality of wearable devices 104. In this instance, the server 106 receives instructions from a governing authority 302 in which the issued commands of the server 106 correspond to governing settings 308 transmitted from the governing authority 302 and received by the server 106.

The governing authority 302 can be a governing body or organization which controls guideline parameters of the wearable device system 100. The governing authority 302 can implement data 304 and rules 306 to define governing settings 308 which update the server 106 with relevant information. As such, guidance information can be determined from the governing settings 308 via the data 304 and rules 306 imposed by the corresponding governing authority 302. When the server 106 is in communication with the wearable devices 104, the server 106 can be configured to update the guidance information and any warning messages as the governing settings are obtained by the server 106. For example, if an accident or a disaster incident has occurred in the proximity of a wearable device 104, the server 106 can update the wearable device 104 with such information based on the governing settings 308.

In some aspects of the present disclosure, the server 106 is in communication with an adaptive feedback system 310. The adaptive feedback system 310 can be configured to receive transmissions from the server 106. For example, the wearable devices 104 can relay information of a detected incident that the server 106 is presently unaware of. The server 106 can then be configured to send such information to the adaptive feedback system 310 which consequently updates the governing settings 308 with the newly received information that was detected at the wearable devices 104. The governing settings 308 may then provide the server 106 with newly updated guidance information and warning messages based on the information from the adaptive feedback system 310 as well as information from the governing authority 302.

The adaptive feedback system 310 enables the server 106 to update the wearable devices 104 with locational information, physiological information, and preferences of the governing authority 302 via the updated governing settings 308. In certain aspects, the wearable device system workflow 300 can function in two modes. The first mode includes a master and slave scenario where the server 106 monitors the wearable devices 104 by sending information, instructions and guidance to the wearable devices 104. The second mode can include an individual scenario in which a first wearable device maintains no connection with a server 106 or other wearable devices initially, but the first wearable device can establish a connection with other wearable devices within a predetermined proximity. In some aspects of the present disclosure, the server 106 can be a master wearable device which issues commands to one or more slave wearable devices.

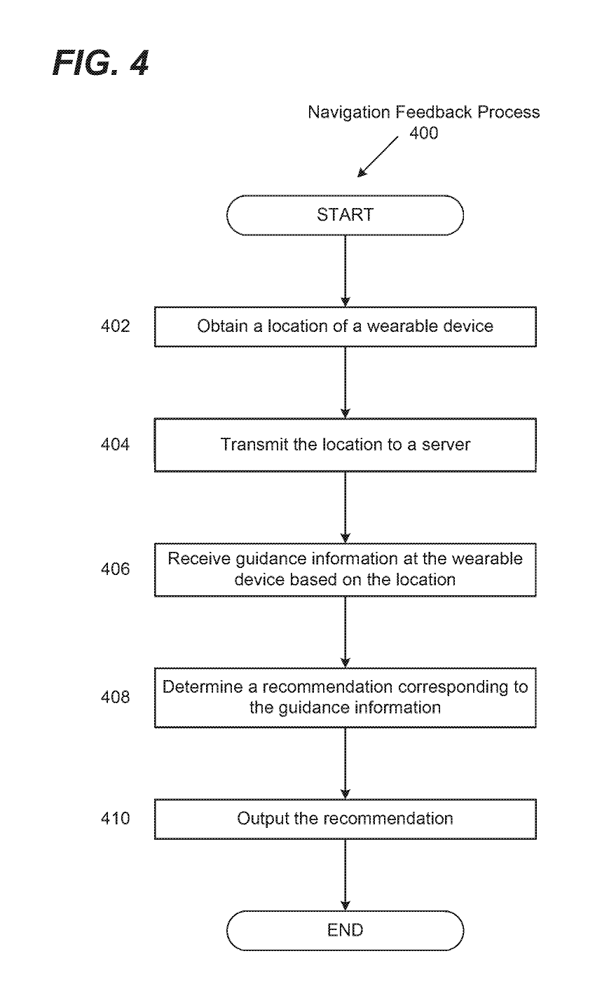

FIG. 4 illustrates a navigation feedback process 400, according to certain exemplary aspects. The navigation feedback process 400 describes a process of establishing communication between a wearable device 104 and a server 106 in which feedback is provided to the wearable device 104 based on data corresponding to detected locational information of the wearable device 104. At step 402, a location of a wearable device 104 is obtained at a locationing system of the wearable device 104 via circuitry of a processor of the wearable device 104. The locationing system 202 can be located in the housing of the wearable device 104. The locationing system 202 can determine an indoor location and an outdoor location of the wearable device 104 via at least one of GNSS, GPS, GIS, GALELIO, Wi-Fi, INS, GSM, and the like.

At step 404, the location of the wearable device 104 is transmitted to the server 106 via the circuitry of the wearable device 104. In some aspects, the location of the wearable device 104 is transmitted to other wearable devices. The location of the wearable device 104 can be transmitted continuously in real time. In other aspects, the location of the wearable device 104 can be transmitted periodically over predetermined intervals of time.

At step 406, the circuitry of the wearable device 104 receives guidance information based on the location. The guidance information can include navigation to a predetermined location, navigation to a desired location that has been input at the graphical display of the wearable device 104 via the tactile interface, and the like. In some aspects, the guidance information can be determined from the governing settings 308 via the data 304 and rules 306 imposed by the corresponding governing authority 302.

At step 408, the circuitry of the wearable device 104 is further configured to determine a recommendation corresponding to the guidance information. The recommendation can include a warning corresponding to an incident that has occurred at the predetermined location and/or the desired location. In other aspects, the recommendation can include directions along a determined path of navigation from the location of the wearable device 104 to the predetermined location or the desired location.

At step 410, the circuitry of the wearable device 104 is configured to output the recommendation. The recommendation can be output visually via the graphical display of the wearable device 104. In other aspects, the recommendation the output can be provided via an output device that is coupled to the housing of the wearable device 104. The output device can include one or more output devices such as an audio jack, a speaker, a haptic device and the like. As such, the recommendation can be output visually, audibly, haptically, or any combination thereof.

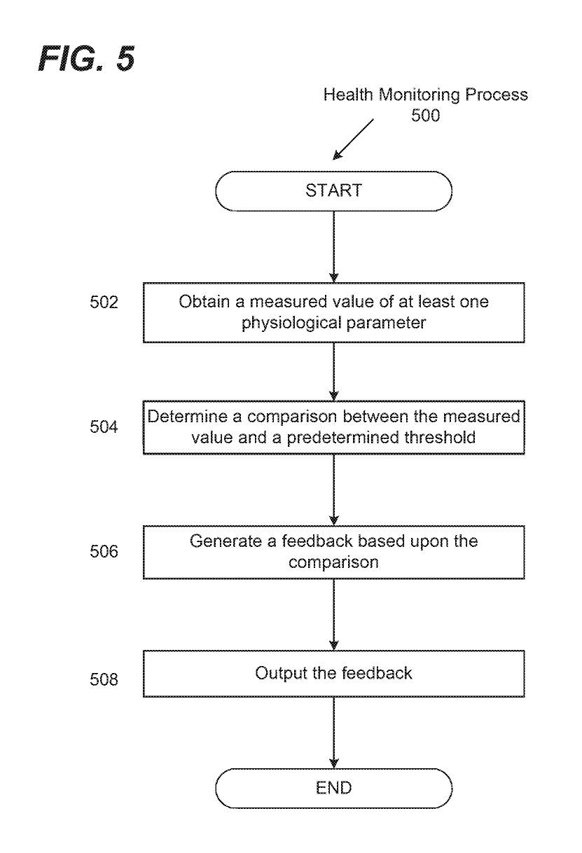

FIG. 5 illustrates a health monitoring process 500, according to certain exemplary aspects. The health monitoring process 500 describes a process of establishing communication between a wearable device 104 and a server 106 in which feedback is provided to the wearable device 104 based on data corresponding to measured physiological information of the wearable device 104. At step 502, a measured value of a physiological parameter is obtained at the wearable device 104. The measured value can include one or more measured values corresponding to one or more physiological parameters. The circuitry of the wearable device 104 can receive the physiological information via a sensor located in the housing of the wearable device 104. For example, the sensor can include one or more sensors such as a heart rate sensor, a respiration rate sensor, a temperature sensor, a blood sugar sensor, a blood pressure sensor, and the like.

At step 504, the circuitry of the wearable device 104 is configured to determine a comparison between the measured value of the physiological parameter and a predetermined threshold corresponding to the physiological parameter. The predetermined threshold can be a lower limit, an upper limit and/or a range depending on the corresponding physiological parameter. Further, each physiological parameter can correspond to more than one threshold. For example, a heart rate physiological parameter may have a first threshold indicative of an upper limit of the heart rate and a second threshold indicative of a lower limit of the heart rate. The thresholds may be determined based upon when the corresponding physiological parameter may indicate an abnormal condition, a fatal condition, an emergency situation and the like.

At step 506, the circuitry of the wearable device 104 is configured to generate a feedback based on the comparison between the measured physiological parameter and the corresponding threshold. As such, the measured values of the one or more physiological parameters are compared to the corresponding thresholds. For example, the feedback can include a warning signal based on the comparison of the measured physiological parameter and the corresponding threshold. In this instance, the warning signal can seek to alert the user that their heart rate has reached dangerously high levels and that the user should proceed with caution. In another example, the feedback may be provided when the blood pressure level is out of a range defined by the corresponding threshold. The feedback may be generated at varying frequencies such as one time, intermittently, periodically, or continually. The frequency with which the feedback is generated can be based on the difference between the threshold and the measured value. For example, the measured value may be within the range or bound of the threshold but also approaching the range or bound of the threshold.

At step 508, the circuitry of the wearable device is configured to output the generated feedback. The feedback can include a visual feedback, an auditory feedback, a haptic feedback and the like. The feedback can notify a user via the graphical display of the wearable device 104, via one or more output devices, and the like. The feedback can include visual feedback notifying the user of a determined comparison via the graphical display of the wearable device 104. The feedback can include a haptic feedback notifying the user of a determined comparison via an output device such as a haptic device. The feedback can include an auditory feedback notifying the user of a determined comparison via an output device such as a speaker. In certain aspects of the present disclosure, feedback can be output to the user as the measured value approaches the range or bound of the threshold. Feedback can also be output to the user at increasing frequency as the measured value surpasses the range or bound of the threshold.

The wearable device 104 can be utilized by travelers to comfortably navigate from one location to another utilizing continuous and precise locational feedback. The locational feedback can be provided as recommendations at the wearable device 104 via the implementation of locational systems such as GNSS, GPS, GIS, GALELIO, Wi-Fi, INS, GSM and the like. The wearable device 104 can also include one or more sensors to detect physiological parameters. The wearable device can measure physiological parameters and generate feedback based on a comparison between the measured physiological parameters and corresponding predetermined thresholds. The wearable device 104 can be configured to communicate with a server 106 as well as other wearable devices. As such, the wearable device 104 can be updated in real time according to feedback that is generated based on guidance information and physiological responses.

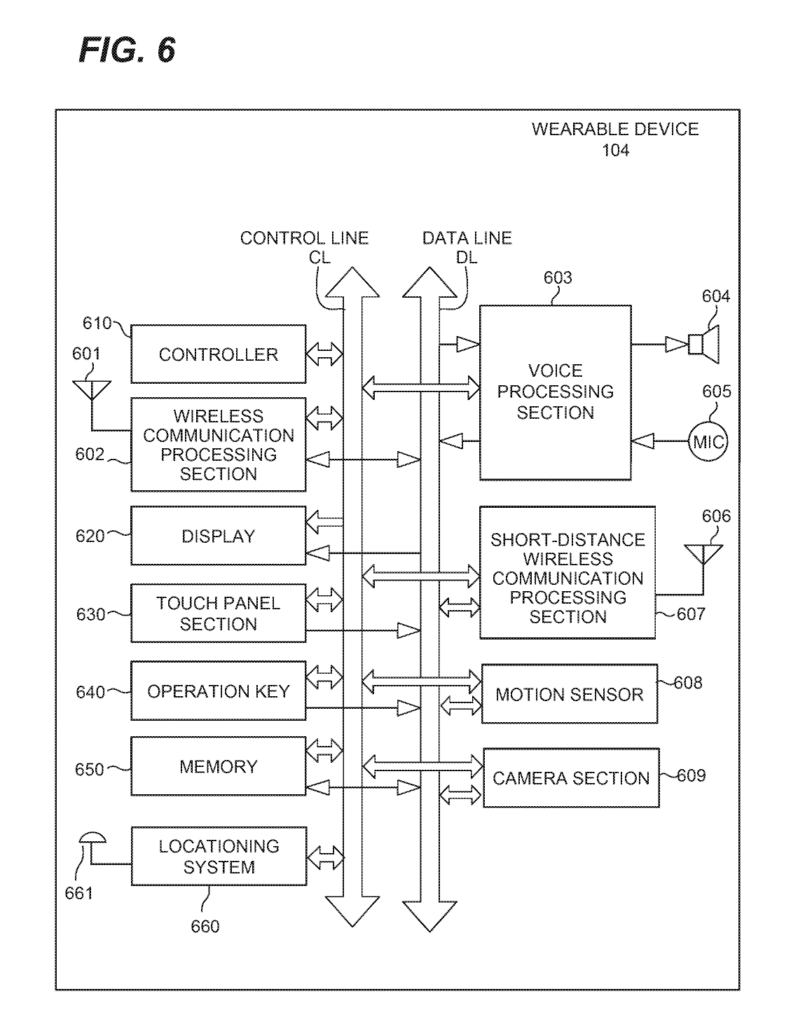

FIG. 6 illustrates a hardware block diagram of a wearable device, according to certain exemplary aspects. FIG. 6 is a more detailed block diagram illustrating an exemplary wearable device 104 according to certain aspects of the present disclosure. In certain aspects, wearable device 104 may be a smart watch or smart wearable device. The exemplary wearable device 104 of FIG. 6 includes a controller 610 and a wireless communication processor 602 connected to an antenna 601. A speaker 604 and a microphone 605 are connected to a voice processor 603.

The controller 610 is an example of the control unit, which may include one or more Central Processing Units (CPUs), and may control each element in the wearable device 104 to perform functions related to communication control, audio signal processing, control for the audio signal processing, still and moving image processing and control, and other kinds of signal processing. The controller 610 may perform these functions by executing instructions stored in a memory 650. Alternatively or in addition to the local storage of the memory 650, the functions may be executed using instructions stored on an external device accessed on a network or on a non-transitory computer readable medium. The controller 610 may execute instructions allowing the controller 610 to function as a display control unit, an operation management unit, game management unit, and the like.

The memory 650 is an example of a storage unit and includes but is not limited to Read Only Memory (ROM), Random Access Memory (RAM), or a memory array including a combination of volatile and non-volatile memory units. The memory 650 may be utilized as working memory by the controller 610 while executing the processes and algorithms of the present disclosure. Additionally, the memory 650 may be used for long-term storage, e.g., of image data and information related thereto.

The wearable device 104 includes a control line CL and data line DL as internal communication bus lines. Control data to/from the controller 650 may be transmitted through the control line CL. The data line DL may be used for transmission of voice data, display data, etc.

The antenna 601 transmits/receives electromagnetic wave signals between base stations for performing radio-based communication, such as the various forms of cellular telephone communication. The wireless communication processor 602 controls the communication performed between the wearable device 104 and other external devices via the antenna 601. For example, the wireless communication processor 602 may control communication between wearable devices for navigation based communication.

The speaker 604 emits an audio signal corresponding to audio data supplied from the voice processor 603. The microphone 605 detects surrounding audio and converts the detected audio into an audio signal. The audio signal may then be output to the voice processor 603 for further processing. The voice processor 603 demodulates and/or decodes the audio data read from the memory 650 or audio data received by the wireless communication processor 602 and/or a short-distance wireless communication processor 607. Additionally, the voice processor 603 may decode audio signals obtained by the microphone 605.

The exemplary wearable device 104 may also include a display 620, a touch panel 630, an operation key 640, and a short-distance communication processor 607 connected to an antenna 606. The display 620 may be a Liquid Crystal Display (LCD), an organic electroluminescence display panel, or another display screen technology. In addition to displaying still and moving image data, the display 620 may display operational inputs, such as numbers or icons which may be used for control of the wearable device 104. The display 620 may additionally display a GUI for a user to control aspects of the wearable device 104 and/or other devices. Further, the display 620 may display characters and images received by the wearable device 104 and/or stored in the memory 650 or accessed from an external device on a network 102. For example, the wearable device 104 may access a network 102 such as the Internet and display text and/or images transmitted from a Web server.

The touch panel 630 may include a physical touch panel display screen and a touch panel driver. The touch panel 630 may include one or more touch sensors for detecting an input operation on an operation surface of the touch panel display screen. The touch panel 630 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 630 may detect when the stylus approaches/contacts the operation surface of the touch panel display (similar to the case in which a finger is used for the touch operation).

One or more of the display 620 and the touch panel 630 are examples of the touch panel display.

In certain aspects of the present disclosure, the touch panel 630 may be disposed adjacent to the display 620 (e.g., laminated) or may be formed integrally with the display 620. For simplicity, the present disclosure assumes the touch panel 630 is formed integrally with the display 620 and therefore, examples discussed herein may describe touch operations being performed on the surface of the display 620 rather than the touch panel 630. However, the skilled artisan will appreciate that this is not limiting.

For simplicity, the present disclosure assumes the touch panel 630 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 630 may include transparent electrode touch sensors arranged in the X-Y direction on the surface of transparent sensor glass.

The touch panel driver may be included in the touch panel 630 for control processing related to the touch panel 630, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in certain embodiments, the touch panel 630 may detect a position of a user's finger around an edge of the display panel 620 (e.g., gripping a protective case that surrounds the display/touch panel). Signals may be transmitted by the touch panel driver, e.g. in response to a detection of a touch operation, in response to a query from another element based on timed data exchange, etc.

The touch panel 630 and the display 620 may be surrounded by a protective casing, which may also enclose the other elements included in the wearable device 104. In certain embodiments, a position of the user's fingers on the protective casing (but not directly on the surface of the display 620) may be detected by the touch panel 630 sensors. Accordingly, the controller 610 may perform display control processing described herein based on the detected position of the user's fingers gripping the casing. For example, an element in an interface may be moved to a new location within the interface (e.g., closer to one or more of the fingers) based on the detected finger position.

Further, in certain aspects, the controller 610 may be configured to detect which hand is holding the wearable device 104, based on the detected finger position. For example, the touch panel 630 sensors may detect a plurality of fingers on the left side of the wearable device 104 (e.g., on an edge of the display 620 or on the protective casing), and detect a single finger on the right side of the wearable device 104. In this exemplary scenario, the controller 610 may determine that the user is holding the wearable device 104 with his/her right hand because the detected grip pattern corresponds to an expected pattern when wearable device 104 is held only with the right hand.

The operation key 640 may include one or more buttons or similar external control elements, which may generate an operation signal based on a detected input by the user. In addition to outputs from the touch panel 630, these operation signals may be supplied to the controller 610 for performing related processing and control. In certain aspects of the present disclosure, the processing and/or functions associated with external buttons and the like may be performed by the controller 610 in response to an input operation on the touch panel 630 display screen rather than the external button, key, etc. In this way, external buttons on the wearable device 104 may be eliminated in lieu of performing inputs via touch operations, thereby improving water-tightness.

The antenna 606 may transmit/receive electromagnetic wave signals to/from other external apparatuses, and the short-distance wireless communication processor 607 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 607.

The wearable device 104 may include a motion sensor 608. The motion sensor 608 may detect features of motion (i.e., one or more movements) of the wearable device 104. For example, the motion sensor 608 may include an accelerometer to detect acceleration, a gyroscope to detect angular velocity, a geomagnetic sensor to detect direction, a geo-location sensor to detect location, etc., or a combination thereof to detect motion of the wearable device 104. In certain embodiments, the motion sensor 608 may generate a detection signal that includes data representing the detected motion. For example, the motion sensor 608 may determine a number of distinct movements in a motion (e.g., from start of the series of movements to the stop, within a predetermined time interval, etc.), a number of physical shocks on the wearable device 104 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 610, whereby further processing may be performed based on data included in the detection signal. The motion sensor 608 can work in conjunction with a locationing system 660. The locationing system 660 detects the present position of the wearable device 104. The information of the present position detected by the locationing system 660 is transmitted to the controller 610. An antenna 661 is connected to the locationing system 660 for receiving and transmitting signals to and from an external locationing device/server.

The wearable device 104 may include a camera section 609, which includes a lens and shutter for capturing photographs of the surroundings around the wearable device 104. In an embodiment, the camera section 609 captures surroundings of an opposite side of the wearable device 104 from the user. The images of the captured photographs can be displayed on the display panel 620. A memory section saves the captured photographs. The memory section may reside within the camera section 609 or it may be part of the memory 650. The camera section 609 can be a separate feature attached to the wearable device 104 or it can be a built-in camera feature.

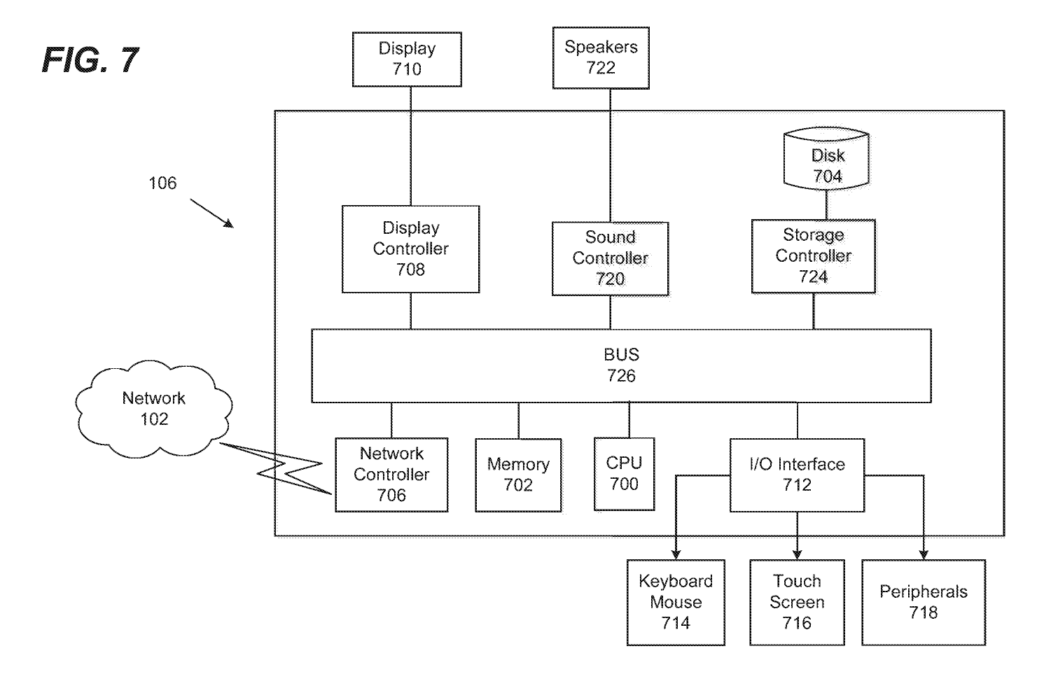

FIG. 7 illustrates a hardware block diagram of a server, according to certain exemplary aspects. In FIG. 7, the server 106 includes a CPU 700 which performs the processes described above/below. The process data and instructions may be stored in memory 702. These processes and instructions may also be stored on a storage medium disk 704 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the server 106 communicates, such wearable device 104 or a computer.

Further, the claimed advancements may be provided as a utility application, background daemon, or component of an operating system, or combination thereof, executing in conjunction with CPU 700 and an operating system such as Microsoft Windows 7, UNIX, Solaris, LINUX, Apple MAC-OS and other systems known to those skilled in the art.

The hardware elements in order to achieve the server 106 may be realized by various circuitry elements, known to those skilled in the art. For example, CPU 700 may be a Xenon or Core processor from Intel of America or an Opteron processor from AMD of America, or may be other processor types that would be recognized by one of ordinary skill in the art. Alternatively, the CPU 700 may be implemented on an FPGA, ASIC, PLD or using discrete logic circuits, as one of ordinary skill in the art would recognize. Further, CPU 700 may be implemented as multiple processors cooperatively working in parallel to perform the instructions of the inventive processes described above.

The server in FIG. 7 also includes a network controller 706, such as an Intel Ethernet PRO network interface card from Intel Corporation of America, for interfacing with network 102. As can be appreciated, the network 102 can be a public network, such as the Internet, or a private network such as an LAN or WAN network, or any combination thereof and can also include PSTN or ISDN sub-networks. The network 102 can also be wired, such as an Ethernet network, or can be wireless such as a cellular network including EDGE, 3G and 4G wireless cellular systems. The wireless network can also be Wi-Fi, BLUETOOTH, or any other wireless form of communication that is known.

The server 106 further includes a display controller 708, such as a NVIDIA GeForce GTX or Quadro graphics adaptor from NVIDIA Corporation of America for interfacing with display 710, such as a Hewlett Packard HPL2445w LCD monitor. A general purpose I/O interface 712 interfaces with a touch screen panel 716 on or separate from display 710. General purpose I/O interface also connects to a variety of peripherals 718 including printers and scanners, such as an OfficeJet or DeskJet from Hewlett Packard.

A sound controller 720 is also provided in the server 106, such as Sound Blaster X-Fi Titanium from Creative, to interface with speakers/microphone 722 thereby providing sounds and/or music.

The general purpose storage controller 724 connects the storage medium disk 704 with communication bus 726, which may be an ISA, EISA, VESA, PCI, or similar, for interconnecting all of the components of the server 106. A description of the general features and functionality of the display 710, as well as the display controller 708, storage controller 724, network controller 706, sound controller 720, and general purpose I/O interface 712 is omitted herein for brevity as these features are known.

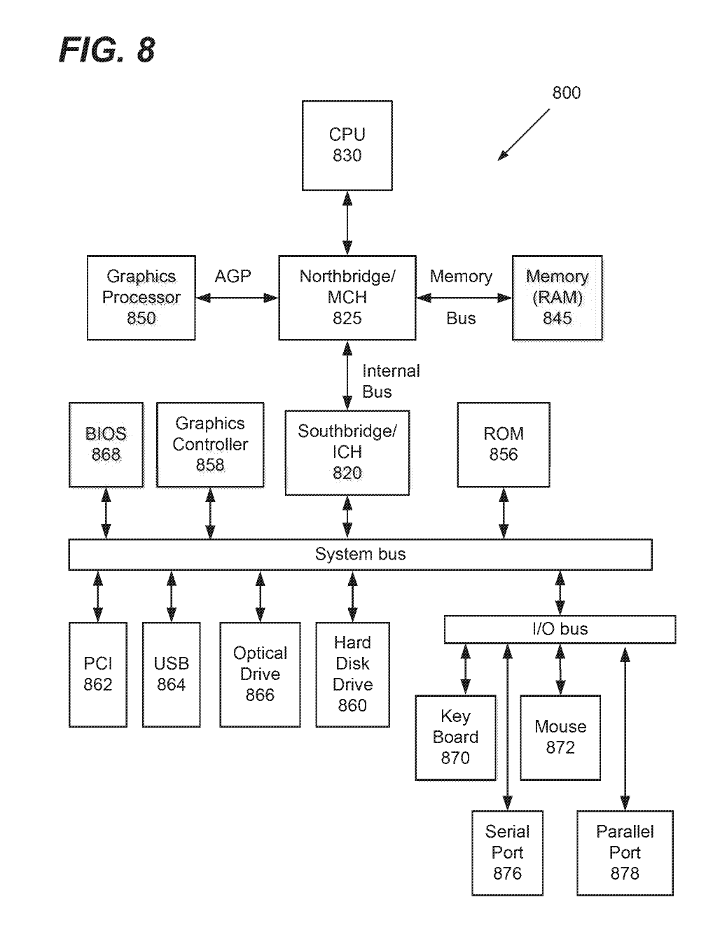

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 8.

FIG. 8 illustrates a hardware block diagram of a data processing system 800, according to certain exemplary aspects of the present disclosure. The data processing system is an example of a computer in which code or instructions implementing the processes of the illustrative aspects may be located.

In FIG. 8, the data processing system 800 employs a hub architecture including a north bridge and memory controller hub (NB/MCH) 825 and a south bridge and input/output (I/O) controller hub (SB/ICH) 820. The central processing unit (CPU) 830 is connected to NB/MCH 825. The NB/MCH 825 also connects to the memory 845 via a memory bus, and connects to the graphics processor 850 via an accelerated graphics port (AGP). The NB/MCH 825 also connects to the SB/ICH 820 via an internal bus (e.g., a unified media interface or a direct media interface). The CPU Processing unit 830 may contain one or more processors and even may be implemented using one or more heterogeneous processor systems.

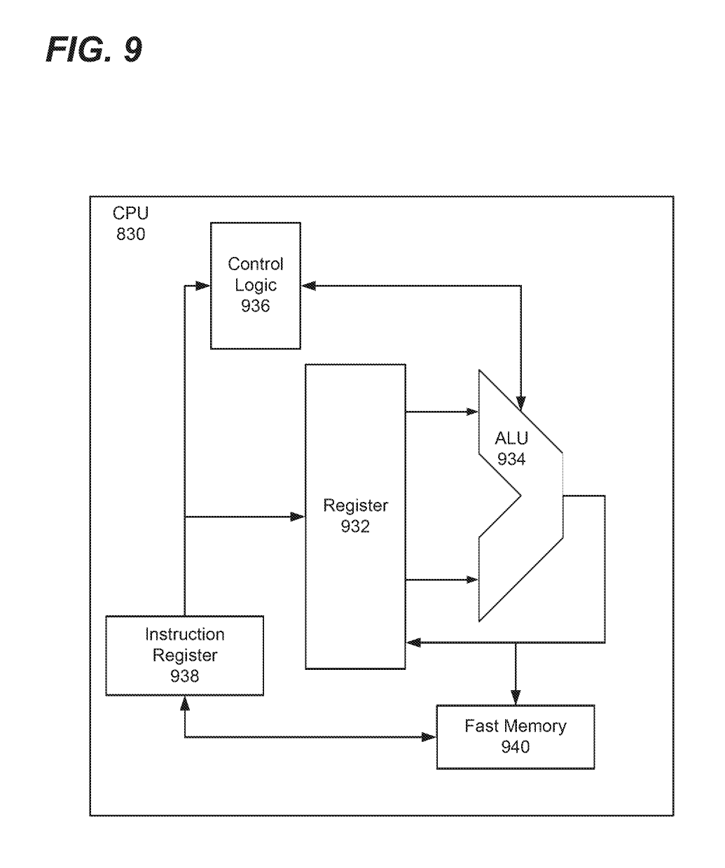

FIG. 9 illustrates a hardware block diagram of a CPU, according to certain exemplary aspects of the present disclosure. For example, FIG. 9 shows one implementation of CPU 830. In one implementation, the instruction register 938 retrieves instructions from the fast memory 940. At least part of these instructions are fetched from the instruction register 938 by the control logic 936 and interpreted according to the instruction set architecture of the CPU 830. Part of the instructions can also be directed to the register 932. In one implementation the instructions are decoded according to a hardwired method, and in another implementation the instructions are decoded according to a microprogram that translates instructions into sets of CPU configuration signals that are applied sequentially over multiple clock pulses.

After fetching and decoding the instructions, the instructions are executed using the arithmetic logic unit (ALU) 934 that loads values from the register 932 and performs logical and mathematical operations on the loaded values according to the instructions. The results from these operations can be feedback into the register and/or stored in the fast memory 940. According to certain implementations, the instruction set architecture of the CPU 830 can use a reduced instruction set architecture, a complex instruction set architecture, a vector processor architecture, a very large instruction word architecture. Furthermore, the CPU 830 can be based on the Von Neuman model or the Harvard model. The CPU 830 can be a digital signal processor, an FPGA, an ASIC, a PLA, a PLD, or a CPLD. Further, the CPU 830 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 8, the data processing system 800 can include that the SB/ICH 820 is coupled through a system bus to an I/O Bus, a read only memory (ROM) 856, universal serial bus (USB) port 864, a flash binary input/output system (BIOS) 868, and a graphics controller 858. PCI/PCIe devices can also be coupled to SB/ICH YYY through a PCI bus 862.

The PCI devices may include, for example, Ethernet adapters, add-in cards, and PC cards for notebook computers. The Hard disk drive 860 and CD-ROM 866 can use, for example, an integrated drive electronics (IDE) or serial advanced technology attachment (SATA) interface. In one implementation the I/O bus can include a super I/O (SIO) device.

Further, the hard disk drive (HDD) 860 and optical drive 866 can also be coupled to the SB/ICH 820 through a system bus. In one implementation a parallel port 878 and a serial port 876 can be connected to the system bust through the I/O bus. Other peripherals and devices that can be connected to the SB/ICH 820 using a mass storage controller such as SATA or PATA, an Ethernet port, an ISA bus, a LPC bridge, SMBus, a DMA controller, and an Audio Codec.

The functions and features described herein may also be executed by various distributed components of a system. For example, one or more processors may execute these system functions, wherein the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process.

The above-described hardware description is a non-limiting example of corresponding structure for performing the functionality described herein.

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. The functions, processes and algorithms described herein may be performed in hardware or software executed by hardware, including computer processors and/or programmable circuits configured to execute program code and/or computer instructions to execute the functions, processes and algorithms described herein. Additionally, an implementation may be performed on modules or hardware not identical to those described. Accordingly, other implementations are within the scope that may be claimed.

The above disclosure also encompasses the aspects listed below.

(1) A wearable device, including: a fastening device to fasten the wearable device to a user, a housing coupled to the fastening device, wherein the housing includes a graphical display including a tactile user interface, at least one locationing system, communication circuitry, at least one sensor for measuring at least one physiological parameter of the user, and a processing circuitry, the processor circuitry being configured to: obtain, via the at least one locationing system, a location of the wearable device, transmit, via the communication circuitry, the location of the wearable device to a server, receive guidance information from the server based on the location of the wearable device, determine a recommendation corresponding to the guidance information, output, via the graphical display, the recommendation, obtain, via the at least one sensor, a measured value of the at least one physiological parameter, determine a comparison between the measured value and a predetermined threshold of the at least one physiological parameter, generate a feedback based upon the comparison, and output, via the graphical display, the feedback.

(2) The wearable device according to (1), wherein the at least one locationing system includes at least one of Global Navigation Satellite Systems (GNSS), Global Positioning System (GPS), Wi-Fi, GALELIO, Geographic Information Systems (GIS), Global System for Mobile Communications (GSM) and Inertial Navigation System (INS).

(3) The wearable device according to either (1) or (2), wherein the processing circuitry is further configured to determine at least one of an indoor location and an outdoor location of the wearable device.

(4) The wearable device according to any one of (1) to (3), wherein the at least one sensor includes at least one of a heart rate sensor, a respiration rate sensor, a temperature sensor, a blood sugar sensor and a blood pressure sensor.

(5) The wearable device according to any one of (1) to (4), wherein the processing circuitry is further configured to store the location of the wearable device and the measured value of the at least one physiological parameter.

(6) The wearable device according to any one of (1) to (5), wherein the guidance information includes at least one of navigation to a predetermined location and navigation to a desired location, the desired location corresponding to a location input at the wearable device via the tactile user interface.

(7) The wearable device according to any one of (1) to (6), wherein the recommendation based on the guidance information includes at least one of a warning corresponding to an event at a predetermined location and a warning corresponding to an event at a desired location, the desired location corresponding to a location input at the wearable device via the tactile user interface.

(8) The wearable device according to any one of (1) to (7), wherein the processing circuitry is further configured to transmit a warning to the server based upon the comparison in which the at least one physiological parameter does not satisfy a condition of the predetermined threshold.

(9) The wearable device according to any one of (1) to (8), further including: one or more output devices coupled to the housing.

(10) The wearable device according to any one of (1) to (9), wherein the one or more output devices include at least one of an audio jack, a speaker and a haptic device.

(11) A method of interacting with a wearable device, including: obtaining, via at least one locationing system of a wearable device, a location of the wearable device; transmitting, via communication circuitry of the wearable device, the location of the wearable device to a server; receiving, via the communication circuitry, guidance information from the server based on the location of the wearable device; determining, via processing circuitry of the wearable device, a recommendation corresponding to the guidance information; outputting, via a graphical display of the wearable device, the recommendation; obtaining, via at least one sensor of the wearable device, a measured value of at least one physiological parameter; determining, via the processing circuitry, a comparison between the measured value and a predetermined threshold of the at least one physiological parameter; generating, via the processing circuitry, a feedback based upon the comparison; and outputting, via the graphical display, the feedback.

(12) The method of (11), wherein the at least one locationing system includes at least one of Global Navigation Satellite Systems (GNSS), Global Positioning System (GPS), Wi-Fi, GALELIO, Geographic Information Systems (GIS), Global System for Mobile Communications (GSM) and Inertial Navigation System (INS).

(13) The method of either (11) or (12), further including: determining at least one of an indoor location and an outdoor location of the wearable device.

(14) The method of any one of (11) to (13), wherein the at least one sensor includes at least one of a heart rate sensor, a respiration rate sensor, a temperature sensor, a blood sugar sensor and a blood pressure sensor.

(15) The method of any one of (11) to (14), further including: storing the location of the wearable device and the measured value of the at least one physiological parameter.

(16) The method of any one of (11) to (15), wherein the guidance information includes at least one of navigation to a predetermined location and navigation to a desired location, the desired location corresponding to a location input at the wearable device via the tactile user interface.

(17) The method of any one of (11) to (16), wherein the recommendation based on the guidance information includes at least one of a warning corresponding to an event at a predetermined location and a warning corresponding to an event at a desired location, the desired location corresponding to a location input at the wearable device via the tactile user interface.

(18) The method of any one of (11) to (17), further including: transmitting a warning to the server based upon the comparison in which the at least one physiological parameter does not satisfy a condition of the predetermined threshold.

(19) A non-transitory computer-readable medium having computer-readable instructions stored therein that when executed by a computer causes the computer to perform a method of interacting with a wearable device, the method including: obtaining a location of the wearable device; transmitting the location of the wearable device to a server; receiving guidance information from the server based on the location of the wearable device; determining a recommendation corresponding to the guidance information; outputting the recommendation to a graphical display; obtaining, via at least one sensor of the wearable device, a measured value of at least one physiological parameter; determining a comparison between the measured value and a predetermined threshold of the at least one physiological parameter; generating a feedback based upon the comparison; and outputting, via the graphical display, the feedback.

(20) The non-transitory computer-readable medium according to (19), wherein the recommendation based on the guidance information includes at least one of a warning corresponding to an event at a predetermined location and a warning corresponding to an event at a desired location, the desired location corresponding to a location input at the wearable device via the tactile user interface.

1. A wearable device, comprising:
a fastening device to fasten the wearable device to a user;
a housing coupled to the fastening device, wherein the housing includes a graphical display including a tactile user interface, at least one locationing system, communication circuitry, at least one sensor for measuring at least one physiological parameter of the user, and a processing circuitry,
the processor circuitry being configured to:
obtain, via the at least one locationing system, a location of the wearable device,
transmit, via the communication circuitry, the location of the wearable device to a server,
receive guidance information from the server based on the location of the wearable device,
determine a recommendation corresponding to the guidance information,
output, via the graphical display, the recommendation,
obtain, via the at least one sensor, a measured value of the at least one physiological parameter,
determine a comparison between the measured value and a predetermined threshold of the at least one physiological parameter,
generate a feedback based upon the comparison, and
output, via the graphical display, the feedback.

2. The wearable device according to claim 1, wherein the at least one locationing system includes at least one of Global Navigation Satellite Systems (GNSS), Global Positioning System (GPS), Wi-Fi, GALELIO, Geographic Information Systems (GIS), Global System for Mobile Communications (GSM) and Inertial Navigation System (INS).

3. The wearable device according to claim 1, wherein the processing circuitry is further configured to determine at least one of an indoor location and an outdoor location of the wearable device.

4. The wearable device according to claim 1, wherein the at least one sensor includes at least one of a heart rate sensor, a respiration rate sensor, a temperature sensor, a blood sugar sensor and a blood pressure sensor.

5. The wearable device according to claim 1, wherein the processing circuitry is further configured to store the location of the wearable device and the measured value of the at least one physiological parameter.

6. The wearable device according to claim 1, wherein the guidance information includes at least one of navigation to a predetermined location and navigation to a desired location, the desired location corresponding to a location input at the wearable device via the tactile user interface.

7. The wearable device according to claim 1, wherein the recommendation based on the guidance information includes at least one of a warning corresponding to an event at a predetermined location and a warning corresponding to an event at a desired location, the desired location corresponding to a location input at the wearable device via the tactile user interface.

8. The wearable device according to claim 1, wherein the processing circuitry is further configured to transmit a warning to the server based upon the comparison in which the at least one physiological parameter does not satisfy a condition of the predetermined threshold.

9. The wearable device according to claim 1, further comprising:
one or more output devices coupled to the housing.

10. The wearable device according to claim 9, wherein the one or more output devices include at least one of an audio jack, a speaker and a haptic device.

11. A method of interacting with a wearable device, comprising:
obtaining, via at least one locationing system of a wearable device, a location of the wearable device;
transmitting, via communication circuitry of the wearable device, the location of the wearable device to a server;
receiving, via the communication circuitry, guidance information from the server based on the location of the wearable device;
determining, via processing circuitry of the wearable device, a recommendation corresponding to the guidance information;
outputting, via a graphical display of the wearable device, the recommendation;
obtaining, via at least one sensor of the wearable device, a measured value of at least one physiological parameter;
determining, via the processing circuitry, a comparison between the measured value and a predetermined threshold of the at least one physiological parameter;
generating, via the processing circuitry, a feedback based upon the comparison; and
outputting, via the graphical display, the feedback.

12. The method of claim 11, wherein the at least one locationing system includes at least one of Global Navigation Satellite Systems (GNSS), Global Positioning System (GPS), Wi-Fi, GALELIO, Geographic Information Systems (GIS), Global System for Mobile Communications (GSM) and Inertial Navigation System (INS).

13. The method of claim 11, further comprising:
determining at least one of an indoor location and an outdoor location of the wearable device.

14. The method of claim 11, wherein the at least one sensor includes at least one of a heart rate sensor, a respiration rate sensor, a temperature sensor, a blood sugar sensor and a blood pressure sensor.

15. The method of claim 11, further comprising:
storing the location of the wearable device and the measured value of the at least one physiological parameter.

16. The method of claim 11, wherein the guidance information includes at least one of navigation to a predetermined location and navigation to a desired location, the desired location corresponding to a location input at the wearable device via the tactile user interface.

17. The method of claim 11, wherein the recommendation based on the guidance information includes at least one of a warning corresponding to an event at a predetermined location and a warning corresponding to an event at a desired location, the desired location corresponding to a location input at the wearable device via the tactile user interface.

18. The method of claim 1, further comprising:
transmitting a warning to the server based upon the comparison in which the at least one physiological parameter does not satisfy a condition of the predetermined threshold.

19. A non-transitory computer-readable medium having computer-readable instructions stored therein that when executed by a computer causes the computer to perform a method of interacting with a wearable device, the method comprising:
obtaining a location of the wearable device;
transmitting the location of the wearable device to a server;
receiving guidance information from the server based on the location of the wearable device;
determining a recommendation corresponding to the guidance information;
outputting the recommendation to a graphical display;
obtaining, via at least one sensor of the wearable device, a measured value of at least one physiological parameter;
determining a comparison between the measured value and a predetermined threshold of the at least one physiological parameter;
generating a feedback based upon the comparison; and
outputting, via the graphical display, the feedback.

20. The non-transitory computer-readable medium according to claim 19, wherein the recommendation based on the guidance information includes at least one of a warning corresponding to an event at a predetermined location and a warning corresponding to an event at a desired location, the desired location corresponding to a location input at the wearable device via the tactile user interface.
